(19) United States
(12) Patent Application Publication
(10) Pub. No.: US 2023/0025033 A1
(43) Pub. Date: Jan. 26, 2023

(54) DENTAL IMPLANT IDENTIFICATION SYSTEM

(71) Applicant: Naval Medical Research Center, Silver Spring, MD (US)

(72) Inventor: Evan Black, Antioch, IL (US)

(73) Assignee: United States of American as Represented by the Secretary of the Navy, Silver Spring, MD (US)

(21) Appl. No.: 17/872,337

(22) Filed: Jul. 25, 2022

Related U.S. Application Data
(60) Provisional application No. 63/225,784, filed on Jul. 26, 2021.

(51) Int. Cl. A61C 8/00; A61B 90/90

(52) U.S. Cl. CPC A61C 8/005; A61C 8/0075; A61B 90/90

(57) ABSTRACT

A dental implant identification system is provided that aids a dental professional in identifying the type of dental implant that has been surgically placed into maid lary or mandibular bone of a patient who requires restorative dental procedures. Additionally, a dental implant identification system is provided. that aids a dental professional in identifying the forensic remains of victims due to various factors and circumstances.

### CROSS-REFERENCE TO RELATED APPLICATIONS

This application claims priority to U.S. Provisional Application No. 63/225,784, filed on Jul. 26, 2021, which is hereby incorporated by reference.

### TECHNICAL FIELD

The subject matter described herein relates to devices, systems, and methods for incorporating one-dimensional (1D), two-dimensional (2D), or three-dimensional (3D) codes or other similar labels or markings in conjunction with the design, manufacture, and use of dental implants. Such devices, systems and methods include codes or labels incorporated into the physical structures of implants, components, and parts, to assist dental implant manufacturers and dental care professionals to track the dental implant throughout the product's life cycle and to be used in forensic identification of a user.

### BACKGROUND OF THE INVENTION

This section provides background information related to the present disclosure, which is not necessarily prior art:

Dental implants are screwed or otherwise inserted into a prepared site in maxillary or mandibular bone and serve as a fixture on which a prosthetic tooth or other dental appliance can be mounted. Dental implants have been widely used in clinical applications and are well known in the field. Since its inception in the 1960s, the use of dental implants has gained significant popularity and exponential growth, resulting in implants of different external and internal features. As a result, dental implants have a variety of diameters, lengths, surfaces, platforms, interfaces, and body designs. While the dental field has benefited significantly from the explosion in dental implant designs, this growth has resulted in uncontrollable variations resulting in incompatibility between different systems and designs. The lack of unified standards for the prosthetic interface poses a serious challenge for the dental professional in terms of serviceability during dental implant restorations. Out of more than 300 implant companies that the inventor personally surveyed, only three companies have tracing features on their implants. These tracing features are used in internal quality controls and are not currently used to aid dental professionals in identifying dental implants.

When the need arises to re-service an existing implant restoration or dental implant, the clinician is often faced with the challenge of identifying the implant, which is a serious problem if clinicians do not have records of the patient's previous treatments. Currently, clinicians often rely on visual examination of radiographic appearance of implant body to identify the implant. Because there is no identifiable information on an existing dental implant, dental professionals are forced to distinguish failed implants through assessment of the physical features of the dental implant, such as its shape, size, thread count, thread design, etc. This process can be very confusing and requires the dentist to have in-depth knowledge of different dental implants on the market, which is an extremely difficult task in which to keep up. Dentists often must reach out to other practitioners personally, or through online blogs and websites, in their attempts to identify a dental implant via radiographs, which is both time consuming and unreliable. Therefore, there is an urgent need for a mechanism for tracking dental implants.

Dental markings have been proposed for personal identification of the patients, and for documentation of features of dental implants and manufacturers. Some methods recommend creating legible markings on the outer surfaces of the dental implants containing identifiable information (FR2970169, KR101298246, US20140302458, US20110076647, and US20190298483). Others suggest affixing an identifiable carrier on the outside of the restoration or near outer surface of the dental implants (U.S. Pat. No. 5,509,805), which can be readily discernable using one of several noninvasive techniques. In some applications, the carrier is marked or labeled with radiopaque coding (U.S. Pat. No. 5,044,955, U.S. RE30594, U.S. Pat. No. 7,357,887) while in other designs the dental carrier is simply a radio frequency identification device (US20090155744). The dental carrier is often inserted into either a naturally occurring or manufactured cavity, which can then be sealed with dental filling to protectively seal the information carrier within the cavity, thus visibly hidden from the external environment.

When information carrying markings or identification carriers that are attached to or inlaid into the surface of the dental implant crown, the information can be easily read using a noninvasive imaging or radio device without the need to first excavate the carrier or the dental implant. Disadvantageously, carriers and marking of this kind are more susceptible to independent or inadvertent removal or destruction. Information carrying markings or carrier on the outside surface of the dental implant crown may be damaged due to normal wear and tear and may be lost due to trauma to the dental implant. If the presence of the markings are not concealed, in the case of a crime, the carrier or markings may be removed by criminals, with the intent to disguise the post-mortem identification of the victim, perhaps by removing the entire dental implant crown. Furthermore, in some applications, in order to identify a corpse or victim of war or mass disaster, the information carrier must be detached from the person, and therefore cannot be used again for confirmatory identification at a later time.

The present disclosure provides a solution for noninvasive identification of dental implants and/or personal identification using concealed dental implant markings. The markings are created at the time of manufacturing of the implant by the implant manufacturer. At a later time, a clinician can use the information contained from the label or marking to identify a dental implant for dental implant restoration, repair, and replacement, and for forensic identification of the patient using various commonly available imaging devices.

This invention is of particular importance for the military, where it can be used to identify service men and women who are left unrecognizable due to unforeseeable war time events or training exercises. Furthermore, because the military uses selected brands of implants, when a military member retires or separates from the military, the civilian dentists who are not familiar with these dental brands will need to be able identify the implants to replace a failed dental implant restoration.

### DETAILED DESCRIPTION OF DRAWINGS

The foregoing and other advantages of the invention will become apparent upon reading the following detailed description and upon reference to the drawings.

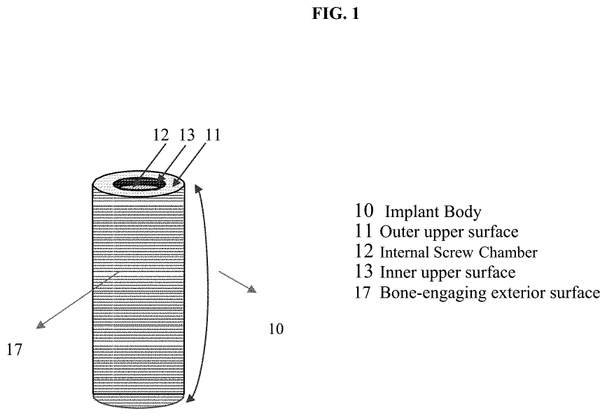

FIG. 1 shows perspective view of a dental implant.

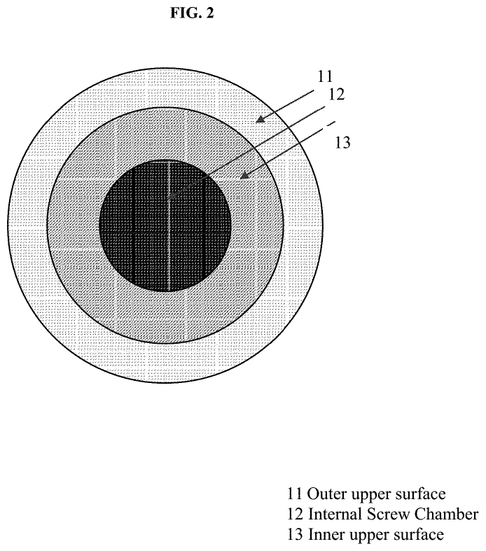

FIG. 2 shows a top view of the upper surface of a dental implant.

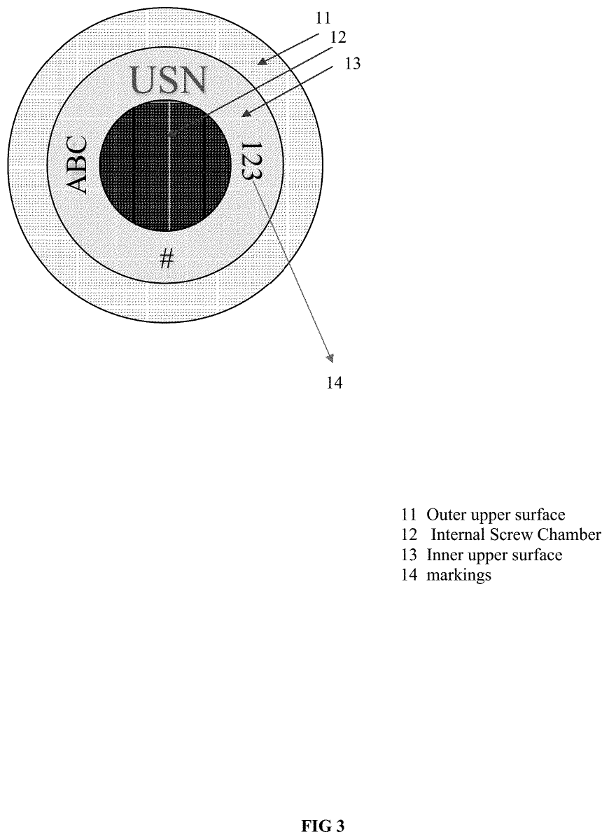

FIG. 3 illustrates a view of the upper surface of a dental implant having a first type of dental marking.

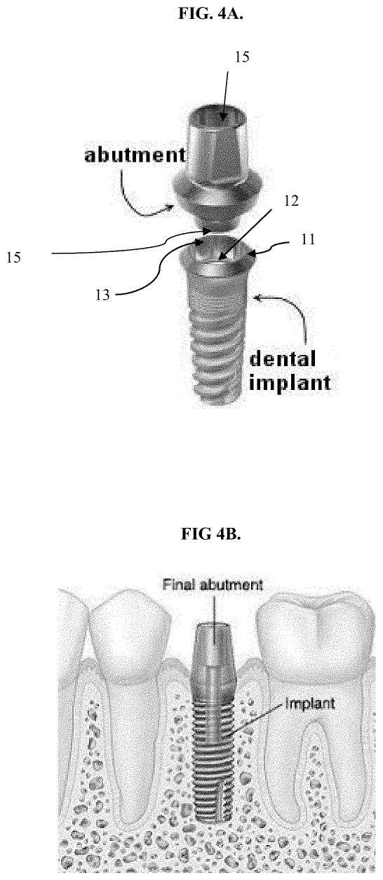

FIG. 4A shows an example abutment and a dental implant before assembly.

FIG. 4B shows an example of abutment and a dental implant in use.

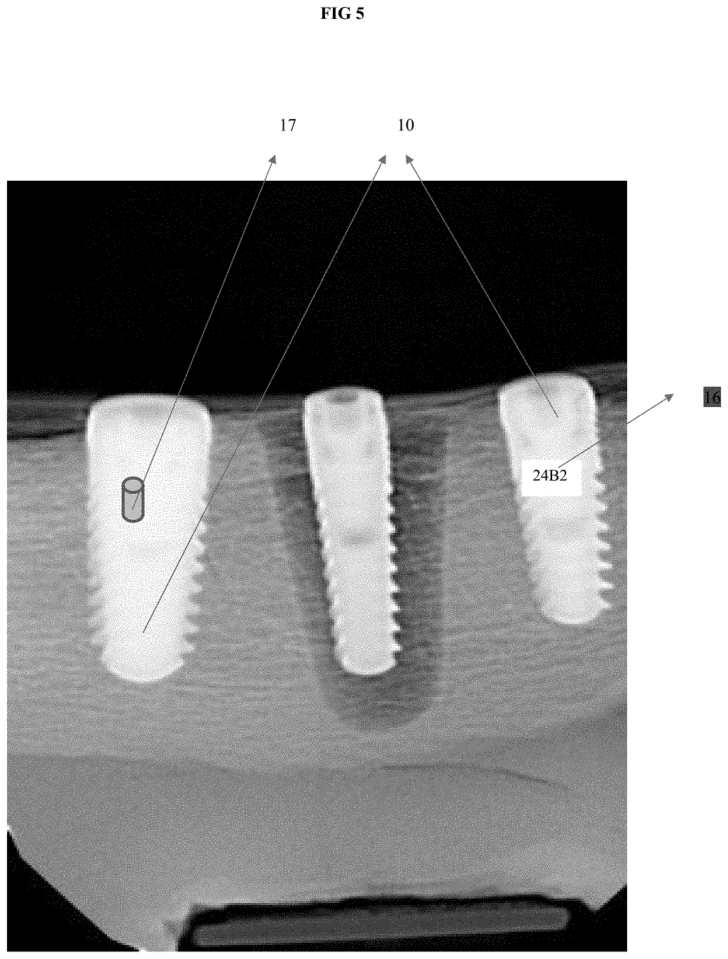

FIG. 5 shows an x-ray photograph of a dental implant having a second type of dental markings.

### SUMMARY OF THE INVENTION

The present invention is directed to the improvements of individual dental implant traceability using a unique set of identifiable markings and the method of using such dental markings to track and identify dental implants or the wearer of the dental implant.

In one aspect, a dental implant for insertion into bone within a patient's mouth comprises an implant body and a visible marking. The implant body includes a bone-engaging exterior surface, an internal screw chamber for mating with an abutment, and an outer upper region. The upper region includes an outer upper surface for engaging the abutment and an inner surface comprising dental marking. The dental markings on the inner upper surface of the dental provides information concerning the dental implant, such as information including but limited to individual serial numbers, lot numbers, batch codes, branding design, date created, manufacturer, models etc. of the dental implant, and/or personal identification information about the patient.

In another aspect, a dental implant for insertion into bone within a patient's mouth comprises an implant body and a radiopaque marking. The body includes a bone-engaging exterior surface, an internal screw chamber for mating with an abutment, an outer upper region. The upper region includes an outer upper surface for engaging the abutment and an inner upper surface for radiopaque marking. The radiopaque marking on the inner upper surface of the dental implant provides information concerning the dental implant, such as information includes individual serial numbers, lot numbers, batch codes, branding design, date created, manufacturer, models etc., and/or personal identification information of the patient.

In another aspect, a dental implant for insertion into bone within a patient's mouth comprises an implant body and a scannable code. The body includes a bone-engaging exterior surface, an internal screw chamber for mating with an abutment, an outer upper region. The upper region includes an outer upper surface for engaging the abutment and an inner upper surface for affixing the scannable code. The scannable code on the inner upper surface provides information concerning the dental implant, such as information including but not limited to individual serial numbers, lot numbers, batch codes, branding design, date created, manufacturer, models etc., and/or personal identification information of the patient.

In yet another aspect, a dental implant for insertion into bone within a patient's mouth comprises an implant body and a marker embedded inside the implant body. The body includes a bone-engaging exterior surface, and an internal screw chamber for mating with an abutment. The upper region includes an outer upper surface for engaging the abutment. The embedded marker may be a 3-dimensional marker that is made of a material of different density than the dental implant, or may be simply a radio frequency identification device. The embedded maker provides information concerning the dental implant, such as information includes individual serial numbers, lot numbers, batch codes, branding design, date created, manufacturer, models etc., or personal identification information of the patient.

In a further aspect, the present invention is a method of using a dental implant that has been placed in bone within the mouth of a patient to identify a dental implant or a patient. The method comprises (i) removing an abutment from a dental implant; (ii) reading the visible marking or scannable code on the inner upper surface of the dental implant; (iii) acquire data corresponding to the visible marking/scannable code on the inner upper surface; and (iv) identifying the dental implant or the patient using acquired data.

In a further aspect, the present invention is a method of using a dental implant that has been placed in bone within the mouth of a patient to identify a dental implant or a patient. The method comprises (i) scanning the upper surface of the dental implant; (ii) acquire scanned data corresponding to the embedded marker or radiopaque marking; and (iii) using the scanned data to identify the dental implant or the patient.

Yet another aspect of the present invention is the placement techniques and processing of such dental markings/markers, including, but not limited to, laser etching, laser engraving, acid/base etchants, paint, color codes, all surface modifications of the titanium or implant material. Unique radiolucent or radiopaque implant material or design that would expose an identifying feature of an unidentifiable implant on a radiograph including, but not limited to serial numbers, lot numbers, batch codes, branding design, bar codes, symbols, engravings. These markings would become apparent based on unique material characteristics and become visible on radiographic imaging when patients initially present to dental offices with failing implant restorations exposed to x-rays.

The above summary is not intended to represent each embodiment or every aspect of the present disclosure. Rather, the summary merely provides an exemplification of some of the novel features presented herein. The above features and advantages, and other features and advantages of the present disclosure, will be readily apparent from the following detailed description of exemplary embodiments and best modes for carrying out the present invention when taken in connection with the accompanying drawings and the appended claims.

### DETAILED DESCRIPTION OF THE INVENTION

While the present disclosure is susceptible to various modifications and alternative forms, specific embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

The present invention is directed to an improvement in dental implant materials or design that would expose identifying features of an unidentifiable implant or allow the identification of a patient via identifiable markings on the dental implant. Term “identifiable markings” refers to one-dimensional (1D), two-dimensional (2D), or three-dimensional (3D) codes or other codes or labels that can be identified using noninvasive dimensional penetrating imaging devices or exploratory devices. In addition, the identifiable markings are fabricated so that it provides information (e.g., identifying, quantitative, and/or qualitative information) about the dental implants that can be read directly or indirectly using noninvasive dimensional penetrating devices.

In general, the identifiable markings are disposed within and/or upon the inner upper surface of an dental implant to provide information that would otherwise be difficult or impossible to convey, determine, or detect without invasive action, harming action to the dental implants and/or the subject (e.g., animal, human, etc.), or impacting osseointegration of that implant is disposed within. By including the identifiable code within the dental implants, the material used to produce the code does not have to be biocompatible. Placement techniques and processing of such markings may include, but are not limited to, laser etching, laser engraving, acid/base etchants, paint, color codes, and surface modifications of the titanium or implant material. To reduce damage of the markings caused by trauma of the dental implants, markings are made to the portion of the dental implants that is internal to the implant and embedded under the gum line/alveolar bone once implanted.

The inventive dental implant has two general designs. In the first design, a visible or scannable dental marking is made on the inner upper surface of the dental implant. When the outer upper surface of the implant is properly restored and intimately engaged with an abutment, the dental marking is protected from normal wear and tear. The dental marking is linked to the implant or patient information stored in one or more databases, similar to surgically implanted Orthopaedic Devices. In a second design, a dental marker is placed inside the implant wherein the 3-dimensional marker is a radio identification device, or made of a material of different density than the dental implant. Using one of the commonly used noninvasive dimensional penetrating imaging devices, such as an X-ray machine, the code can be read off the dental marker and linked to dental implant information or patient information stored in one or more databases.

Referring to FIG. 1 and FIG. 4A, a dental implant 10 with a generally elongated body and a bore, wherein said bore mates and attaches to an abutment. An abutment is a connecting element used to attach a crown, bridge, or removable denture to the dental implant fixture. The bore is often partially threaded forming an internal screw chamber 12. The abutment attaches to the dental implant 10 by an abutment or a cover screw (not shown), which advance through the abutment via a central bore 15 and engages the threads inside the internal screw chamber 12 of the dental implant. However, other types of anti-rotational features (both internal and external) can be also used to secure the abutment to the dental implant 10.

Referring to FIG. 2-4A, the upper portion of a dental implant 10 further comprises an outer upper surface 11, which engages the abutment when the abutment is attached to the dental implant 10. The upper portion of the dental implant 10 also comprises a depressed or dented inner upper surface 13, which can be flat or concave. The inner upper surface 13 of the dental implant 10 is hidden between the dental implant and abutment, and thus protected from wear and tear after placement inside the patient's mouth. In an embodiment of present invention, visible markings 14 may be made on the inner upper surface 13. These visible markings 14 may be used to identify the dental implant, linked to information including but not limited to individual serial numbers, lot numbers, batch codes, branding design, date created, manufacturer, models etc. The visible markings 14 may be also used to identify the patient, linked to personal information about the patient, including but not limited to patient name, gender, age, race, dental clinic, or dentist performed the procedure, and date of the procedure etc. The visible dental markings 14 may include, but are not limited to, numbers and alphabetical letters of different fonts, sizes, colors or bar codes and symbols. A variety of known techniques may be used to create visible dental markings including, but are not limited to, laser etching, laser engraving, acid/base etchants, paint, and all other surface modifications techniques of the titanium and other dental implant material.

To identify a patient or a dental implant using visible dental implant markings of the present invention, a clinician must first remove the abutment from the dental implant to reveal the inner upper surface of the dental implant with a universal driver. The clinician reads the visible marking or scannable code on the inner upper surface of the dental implant manually or using an electric reader. The acquired information (i.e. visible marking or scannable code) is then input into one or more databases linking the dental marking or code to the implant information and/or patient information. The clinician can then use the information to identify the dental implant used or the patient. Although a national database for dental implants is not yet established, similar databases exist for other medical implants, such as databases for medical orthopedic devices, and breast implants. Dental implants are medical devices, therefore a national database for dental implants can be easily established to allow dental implant registration and tracking.

In an alternative embodiment, the inner upper surface 13 may have a radiopaque or radiolucent marking. The radiopaque marking on the inner upper surface 13 of the dental implant 10 can be linked to information concerning the dental implant, such as information includes individual serial numbers, lot numbers, batch codes, branding design, date created, manufacturer, models etc., and/or linked to personal information about the patient, The clinician can then use the information to identify the dental implant used or the patient. These radiopaque markings may include, but are not limited to, numbers and alphabetical letters of different fonts, sizes or colors, bar codes, and symbols etc. The radiopaque markings may be created by several known techniques, such as coating the inner upper surface 13 of the dental implant 10 with radiopaque or radiolucent materials. These markings thus will become become visible on radiographic images, such as x-rays taken on patients with failing implant restorations.

Referring to FIG. 5, an alternative design of the present invention, a dental implant for insertion into bone within a patient's mouth comprises an implant body 10 and a marker 17 or 16 that is embedded inside the implant body. The implant body 10 includes a bone-engaging exterior surface 17, and an internal screw chamber 12 for mating and attachment to an abutment via an abutment screw or a cover screw (not shown). The upper region of the implant includes an outer upper region for engaging the abutment (See FIG. 4A-B). The embedded marker 17, 16 may be a radio frequency identification device 16 or a radiopaque or radiolucent marker 17 that is made of a material, which has different density than the dental implant. The embedded marker 17 or 16 provides a code, which can be linked to information concerning the dental implant, such as information including but not limited to individual serial numbers, lot numbers, batch codes, branding design, date created, manufacturer, models etc., or personal information about a patient including, but not limited to: serial numbers, lot numbers, batch codes, branding design, bar codes, symbols, engravings. The radiopaque marker can be a 2- or 3-dimentional marker, which become apparent based on unique material characteristics and on radiographic imaging when patients initially present to dental offices with failing implant restorations exposed to x-rays.

A three-dimensional radiopaque marker may be an object disposed within the dental implant, whose surface is coated with one or more contrast enhancing materials viewable via an noninvasive dimensional penetrating imaging device. The contrast enhancing material can include contrast media such as X-ray contrast media and magnetic resonance contrast media. X-ray contrast media is used to alter the contrast in X-rays (e.g., increase or decrease the attenuation of the X-rays). Positive X-ray contrast media increases the attenuation of the X-rays and can include, but are not limited to, iodinated contrast media and barium contrast media. Iodinated contrast media can include, but is not limited to, water insoluble iodinated media (e.g., propyliodone compounds), oily contrast media (e.g., lipiodol compounds and iodophenyllundecyclic compounds mixed in an oil), and water-soluble iodinated media (e.g., mono-/poly-iodinated pyridone compounds and tri-iodinated benzene ring compounds (monomeric-/dimeric). Magnetic resonance contrast media can include positive and negative contrast media. Positive contrast magnetic resonance media can include small molecular weight compounds containing gadolinium (Gd), manganese (Mn), and iron (Fe), superparamagnetic contrast media, and perfluorinated contrast media. For example, the media can include, but is not limited to, gadopentate meglumine compounds, gadodiamide, gadoversetamide, ferric ammonium citrate, gadoxetic acid, diphenylcyclohexyl phosphodiester, manganese substituted hydroxylapatite PEG-APD and the like.

Negative contrast magnetic resonance media include, but is not limited to, sprodyamide, ferrum oxide, carboxy-dextran coated iron oxide nanoparticles, micro-/poly-crystalline iron oxide nanoparticles, perfluorooctylbromide, barium suspensions and clay mineral particles, and the like.

In another embodiment, the contrast enhancing material could be part of and/or combined with the build material, binder material, and/or other components used to fabricate the dental implants, so long as it doesn't impact osseointegration. For example, two different contrast enhancing materials can be used for the marker and the build/binder material. The marker thus will be visible using the same or different noninvasive dimensional penetrating imaging device. The two different contrast enhancing materials can be used to make different portions of the dental implants (e.g., adjacent or embedded within one another). For example, one identifiable structure can be embedded within another identifiable structure, where each identifiable structure is made of a different contrast enhancing material each of which can be viewed using different noninvasive dimensional penetrating imaging devices.

To identify the patient or a dental implant using a radiopaque or radiolucent marking/marker, or radio frequency identification device, a clinician first scans the patent's implant, including an upper surface of the dental implant, using a noninvasive imaging device or radiofrequency detector. The acquired data is then input into one or more databases capable of linking said dental marking or marker to implant and patient information. A clinician can use the search result can identify the dental implant use or the patient.

Many variations and modifications may be made to the above-described embodiments. It is to be understood that the features of the various embodiments described herein are not mutually exclusive and may exist in various combinations and permutations.

What is claimed is:

1. A dental implant for insertion into bone within a patient's mouth comprises
a) an implant body, with a bone-engaging exterior surface, an internal screw chamber for mating with an abutment, and an upper region, wherein said upper region further comprises an outer surface for engaging the abutment and an inner surface; and
b) a dental marking located on said inner surface of said upper region of said implant body, wherein said dental marking is linked to information about said dental implant and/or said patient.

2. Dental implant of claim 1, wherein said dental implant is information selected from the group consisting of: serial numbers, lot numbers, batch codes, branding design, date created, manufacturer, a models.

3. Dental implant of claim 1, wherein said patient information is information selected from the group consisting of: individual serial number, lot number, batch code, branding design, date created, manufacturer, model, and a combination thereof.

4. Dental implant of claim 1, wherein said patient information includes patient name, gender, age, race, dental clinic, dentist performed the procedure, date of the procedure or a combination thereof.

5. Dental implant of claim 1, wherein said dental marking is a visible dental marking, or radiopaque marking.

6. Dental implant of claim 4, wherein said visible marking is a one-dimensional code, a two-dimensional code or a 3-dimentional code.

7. Dental implant claim 4, wherein said visible marking is made using laser etching, laser engraving, acid/base etchants, paint, color codes, or surface modifications of the titanium or implant material.

8. Dental implant of claim 1, wherein said dental marking is a scannable code.

9. A dental implant for insertion into bone within a patient's mouth comprises
a) an implant body, wherein said implant body comprises
(i) a bone-engaging exterior surface;
(ii) an internal screw chamber for mating with an abutment; and
(iii) an upper region for engaging the abutment, and
b) a marker embedded inside the implant body.

10. The dental implant of claim 9, wherein said embedded marker is a radiopaque marker that is made of a material of different density than the dental implant, or a radio frequency identification device.

11. Dental implant of claim 9, wherein said embedded marker is linked to dental implant information or personal information about the patient.

12. Dental implant of claim 11, wherein said dental implant information is information selected from the group consisting of serial numbers, lot numbers, batch codes, branding design, date created, manufacturer, models or a combination thereof.

13. Dental implant of claim 11, wherein said patient information includes patient name, gender, age, race, dental clinic, dentist performed the procedure, date of the procedure or a combination thereof.

14. Dental implant of claim 10, wherein said radiopaque marker is a 2-dimentional marker or a 3-dimentional marker.

15. A method identify a dental implant or a patient via a dental implant of claims 1-8, comprising
a) removing an abutment from a dental implant;
b) reading a visible marking or a scannable code on an inner upper surface of the dental implant;
c) acquiring data contained in the visible marking or the scannable code by searching in one more databases, which links implant marking or code to implants or patient information; and
d) identifying the dental implant or the patient using said acquired data.

16. A method identify a dental implant or a patient via a dental implant of claims 9-14, comprising:
a) scanning the mouth of a patient including an upper surface of a dental implant;
b) acquiring data contained in an embedded marker or a radiopaque marking on the dental implant;
c) acquiring data contained in the visible marking or the scannable code by searching in one more databases, which links implant marking or code to implants or patient information; and
d) identifying the dental implant or the patient using said acquired data.
